(19) United States
(12) Patent Application Publication
(10) Pub. No.: US 2007/0059773 A1
(43) Pub. Date: Mar. 15, 2007

(54) Identification of therapeutic compounds

(76) Inventor: Peter Richardson, Cambridge (GB)

Correspondence Address: FISH & RICHARDSON PC, P.O. BOX 1022, MINNEAPOLIS, MN, 55440-1022, US

(21) Appl. No.: 10/547,462

(22) PCT Filed: Mar. 5, 2004
(86) PCT No.: PCT/GB04/00902

§ 371 Date: Oct. 27, 2006

(30) Foreign Application Priority Data
Mar. 7, 2003 (GB) 0305153.9

(51) Int. Cl. G01N 33/567; G01N 33/53

(52) U.S. Cl. 435/7.2

(57) ABSTRACT

Methods for identifying potential therapeutic agents involve determining the affinity and/or efficacy of a test compound for an adensoine receptor at a relatively high pH and at a relatively low pH. Compounds with greater affinity and/or efficacy at the low pH are identified as potential therapeutic agents, in particular for the treatment of pain or inflammation.

This invention relates to the identification and therapeutic use of compounds that can be targeted to a site requiring therapy.

In normal mammalian tissues extracellular pH is tightly regulated between pH 7.35 and 7.45. Some tissues experience lower pH values, particularly the lumen of the stomach (pH between 2 and 3) and the surfaces of some epithelia (for example, the lung surface pH is approximately 6.8, Jayaraman, S. et al. (2001) Am. J. Physiol. Cell Physiol. 281, C1504-1511).

In pathological tissues, for example during inflammation, ischaemia and other types of damage, a reduction in pH occurs. For instance, in the arthritic joint, reductions in pH of up to 1 unit are typically seen (Andersson, S. E. et al. (1999) J. Rheumatol. 26, 2018-2024). It has recently been shown that cartilage in osteoarthritic joints experiences pH values as low as 5.0 (Konttinnen, Y. T. et al., (2002) Arth. Rheum. 46, 953-960). Excessive neuronal activity can also result in a reduction in extracellular pH. For instance, epileptic discharges and overstimulation of the spinal cord have been associated with changes in extracellular pH (Chesler, M. Prog. Neurobiol. (1990) 34, 401-427). Similarly, reductions in extracellular pH have been observed in solid tumours where pH values as low as 5.5 have been observed, probably as consequence of poor perfusion and the predominance of glycolytic metabolism (Thistlethwaite, A. J. et al. Radiation Oncology Biol. Phys. (1985) 11, 1647-1652). In ischaemia the lack of blood supply results in a fall in pH (Immke, D. C. & McCleskey, E. W. (2001) Nature Neurosci. 4, 869-870) and subsequent inflammatory cell activation.

A common factor in all these diseases and conditions is that the energy demands of the tissues involved outstrip the supply. This results in anaerobic metabolism with the production of lactic and pyruvic acids. Another contributory factor to pH changes is the emptying of secretory vacuoles (for example in nerve endings or inflammatory cells) into the extracellular space, since the contents of these vacuoles are maintained at low pH. The reduction in pH can help to protect the tissue. For example, the NMDA receptor in the CNS, which has been strongly implicated in ischaemic damage, is inhibited at pH 6.8.

Another common factor in the above diseases is adenosine. Adenosine is a ubiquitous local hormone/neurotransmitter that acts on four known receptors, the adenosine A1, A2A, A2B and A3 receptors. Adenosine generally serves to balance the supply and demand of energy in tissues. For example, in the heart released adenosine slows the heart by an A1 receptor mediated action in the nodes and atria (Belardinelli, L & Isenberg, G Am. J. Physiol. 224, H734-H737), while simultaneously dilating the coronary artery to increase energy (i.e. glucose, fat and oxygen) supply (Knabb et al., Circ. Res. (1983) 53, 33-41). Similarly during inflammation adenosine serves to inhibit inflammatory activity, while in conditions of excessive nerve activity (e.g. epilepsy) adenosine inhibits nerve firing (Klitgaard et al., Eur J. Pharmacol. (1993) 242, 221-228). This system, or a variant on it, is present in all tissues. Thus, under conditions where the pH falls, local adenosine serves to balance the energy supply and demand thus restoring normal tissue function and pH.

Adenosine itself can be used to diagnose and treat supraventricular tachycardia. Adenosine A1 receptor agonists are known to act as powerful analgesics (Sawynok, Eur J. Pharmacol. (1998) 347, 1-11). Adenosine A2A receptor agonists are known to act as anti-inflammatory agents (for example, from U.S. Pat. No. 5,877,180 and WO 99/34804). In experimental animals, A2A receptor agonists have been shown to be effective against a wide variety of conditions including sepsis, arthritis, and ischaemia/reperfusion injury arising from renal, coronary or cerebral artery occlusion. The common factor in these conditions is a reduction in the inflammatory response caused by the inhibitory effect of this receptor on most, if not all, inflammatory cells.

However, the ubiquitous distribution of adenosine receptors means that administration of adenosine receptor agonists causes adverse side effects. This has generally precluded the development of adenosine-based therapies. Selective A1 receptor agonists cause bradycardia. The first selective A2A receptor agonist (2-[4-(2-carboxyethyl)phenylethylamino]-5′-N-ethylcarboxamidoadenosine, or CGS21680), was tested in a Phase 2A clinical trial as a potential anti-hypertensive. However, administration caused a large fall in blood pressure and increase in cardiac output. FR2162128 discloses that adenosine derivatives (including 2-alkoxy adenosine derivatives comprising a lower alkyl group of not less than two carbon atoms) have hypotensive and coronary vasodilatory activity.

Bartlett et al (J. Med. Chem. 1981, 24, 947-954) discloses the evaluation of analogues of 1-methylisoguanosine. These analogues include 2-methoxyadenosine (also known as spongosine). This and other compounds were tested for their skeletal muscle-relaxant, hypothermic, cardiovascular and anti-inflammatory effects in rodents following oral administration. 2-methoxyadenosine caused 25% inhibition of carageenan-induced inflammation in rats at 20 mg/kg po. However, reductions in mean blood pressure (41%), and heart rate (25%) were also observed after administration of this compound at this dose.

There is, therefore, a need to provide adenosine receptor agonists that can be administered with minimal side effects.

Askalan and Richardson (J. Neurochem. (1994) 63:1477-1484), describes the role of histidine residues in the adenosine A2A receptor ligand-binding site. In particular, the pH-dependency of the binding of ligands was examined. A two- to four-fold increase in affinity was observed for some ligands on lowering the ambient pH from 7.0 to 5.5. However, the action of adenosine A2A receptors was not studied.

Hiley et al investigated the effects of pH on responses to adenosine in the isolated perfused superior mesenteric arterial bed of the rat. Reducing the pH of the perfusate to 6.8 enhanced the dilator responses to adenosine by 10-fold.

The applicant has now surprisingly found that, at relatively low pH values within the physiological range, some compounds have substantially higher (approximately 100-fold higher) affinity for and/or efficacy of action at adenosine receptors than at higher pH values. It is believed that this is because the receptors change their conformation in response to changes in pH.

The applicant has appreciated that the pH sensitivity of adenosine receptors can be exploited to identify other adenosine receptor ligands that only have high receptor affinity and/or efficacy under conditions of low pH. The actions of such adenosine receptor ligands may then be targeted to regions of low pH, such as pathological tissues, without causing the serious side effects associated with administration of known adenosine receptor ligands.

According to the invention there is provided a method for identifying a potential therapeutic agent, which comprises determining the affinity and/or efficacy of a test compound for an adenosine receptor at a higher pH of at least pH 7.4, and also at a lower pH, from 5.5 to 7.2, and identifying the compound as a said agent if the affinity and/or efficacy at the lower pH is greater than that at the higher pH.

It is preferred that the difference between the affinity of the potential therapeutic agent for, and/or its efficacy of action at, the adenosine receptor at the lower and higher pH is as great as possible. Where there is a large difference in affinity and/or efficacy, it is expected that the agent can be administered at a dosage at which it has beneficial therapeutic effects, and any side effects associated with higher doses of the agent are avoided or minimised. Preferably the affinity and/or efficacy at the lower pH is over 10 times, more preferably over 100 times, most preferably over 1000 times, greater than the affinity and/or efficacy at the higher pH.

Agonists that have higher affinity and/or efficacy at lower pH may be useful in therapy, for example in the prevention, treatment, or amelioration of pain, particularly neuropathic or chronic inflammatory pain and arthritis. The selectivity of these agonists means that they can be targeted to pathological tissues, for example to arthritic joints, thereby allowing effective administration of lower doses than suggested by the prior art, and minimising side effects.

Methods of the invention may be used to identify ligands that only bind to the adenosine A2A receptor with high affinity at low pH, when the receptor is believed to adopt a different conformation from that existing at pH 7.4. As these ligands are only effective agonists at low pH, they may only act at sites within the body where pH is depressed, most notably in the joint capsule of arthritis and at similar inflammatory pain sites.

Methods of the invention may be used to identify ligands that only bind to the adenosine A1 receptor with high affinity at low pH. Such ligands are expected to be useful in reducing excessive tissue activity, for example nervous activity, and to have reduced side effects compared to known adenosine A1 receptor agonists. Side effects of the known agonists include bradycardia and arteritis.

Methods of the invention may be used to identify ligands that only bind to the adenosine A3 receptor with high affinity at low pH. Such ligands are expected to be useful in the treatment of inflammatory disorders.

Based on the information provided herein, one of ordinary skill in the art can identify suitable active agents for the treatment of specific disease states. These agents would be selectively active in the pathological tissue, thus reducing the probability and severity of side effects associated with receptors in normal tissue.

In isolated tissue and cell preparations, pH sensitivity can be measured accurately. Therefore, after evidence has been obtained in animal models of pathology (for example, inflammatory pain), compounds meeting the appropriate criterion will serve as “hits” for subsequent drug discovery.

Compounds identified using methods of the invention are expected to be of use in the therapy, including prophylaxis, of various conditions. These include diseases or conditions in which prevention, treatment, or amelioration of the disease or condition is mediated by activation of adenosine receptors. Examples include diseases or conditions in which local tissue energy demand exceeds supply and/or pH falls, such as inflammation, ischemia-reperfusion injury, excessive neuronal activity (for example in epilepsy, or chronic pain or hyperalgesia, including inflammatory and neuropathic pain), sepsis, septic shock, neurodegeneration (for example Alzheimer's disease), and other conditions where energy demand exceeds supply, for example muscle fatigue or athletes' cramp.

Compounds identified using methods of the invention may be effective in the prevention, treatment, or amelioration of pain, in particular the following types of pain: bowel pain, pancreatic pain, pelvic/perineal pain, back pain, lower back pain, chest pain, cardiac pain, pelvic pain/PID, joint pain (for example, associated with tendonitis, bursitis, acute arthritis), neck pain, obstetric pain (labour or Caesarean-Section), cancer pain, HIV pain, phantom limb pain, post-operative pain, chronic neuropathic pain, failed back surgery pain, post physical trauma pain (including pain caused by a gunshot wound, a road traffic accident, or a burn), scar tissue pain, acute herpes Zoster pain, acute pancreatitis breakthrough pain (cancer), post-herpes neuralgia, trigeminal neuralgia.

Compounds identified using methods of the invention may be effective in the prevention, treatment, or amelioration of neuropathic or other pain caused by, or associated with diabetic neuropathy, polyneuropathy, fibromyalgia, myofascial pain syndrome, osteoarthritis, rheumatoid arthritis, sciatica or lumbar radiculopathy, spinal stenosis, temporo-mandibular joint disorder, renal colic, dysmenorrhoea/endometriosis.

Compounds identified using methods of the invention may be effective in the prevention, treatment, or amelioration of inflammatory or other pain caused by, or associated with arthritic conditions such as osteoarthritis, rheumatoid arthritis, rheumatoid spondylitis, gouty arthritis, or asthma, chronic obstructive pulmonary disease, fibrosis, multiple sclerosis, sepsis, septic shock, endotoxic shock, gram negative shock, toxic shock, hemorrhagic shock, adult respiratory distress syndrome, cerebral malaria, organ transplant rejection, pain secondary to cancer, HIV, chronic pulmonary inflammatory disease, silicosis, pulmonary sarcosis, bone resorption diseases, reperfusion injury, graft v. host rejection, multiple sclerosis, myasthenia gravis, allograft rejections, fever and myalgia due to infection, AIDS related complex (ARC), keloid formation, scar tissue formation, Crohn's disease, ulcerative colitis and pyresis, irritable bowel syndrome, osteoporosis, cerebral malaria, bacterial meningitis, or adverse effects from amphotericin B treatment, interleukin-2 treatment, OKT3 treatment, or GM-CSF treatment.

Compounds identified by a method of the invention may be particularly effective for the prevention, treatment, or amelioration of particular types of inflammation, including arthritis particularly at the joint capsule of arthritis), asthma, psoriasis, and bowel inflammation.

Compounds identified by a method of the invention may be particularly effective in the prevention, treatment, or amelioration of rheumatoid arthritis, irritable bowel syndrome or osteoarthritis.

The utility of a compound may be determined by a radioligand assay described below. The increase in affinity can be measured for the high affinity binding site (i.e. the agonist active site), while the efficacy of the agonist at different pH values can be determined by assessing the difference in the affinity of the low and high affinity states. This can be done using GTP and stable analogues thereof, which convert the high affinity state of the receptor to the low affinity state thus mimicking activation of the receptor by agonists. Ligands with no efficacy (i.e. antagonists) do not differentiate between these different affinity states. Alternatively, agonists that have different affinities and/or efficacies at different pH values can be identified in functional assays, such as measurement of second messenger production or removal (e.g. stimulation or inhibition of cAMP accumulation, inositol phosphate turnover, calcium signalling, kinase activation etc).

The affinity of a test compound for an adenosine receptor may be determined by any of the following methods:

i) binding of multiple concentrations of labelled test compound (including radiolabelled test compound) to adenosine receptors present in tissues, tissue slices, intact cells, disrupted cells or cell derived membranes;

ii) displacement of a bound labelled compound from adenosine receptors by incubation with unlabelled test compound using tissues, tissue slices, intact cells, disrupted cells or cell derived membranes bearing such receptors. The labelled compound may be either the test compound, or a selective ligand for the receptor.

It will be appreciated that the efficacy of an agonist reflects its ability to activate its receptor. Agonists with high efficacy elicit a maximum receptor response, and partial agonists elicit a smaller response. The efficacy of action of a test compound at an adenosine receptor may be determined by any of the following methods:

i) determining adenosine receptor action by the measurement of the accumulation or depletion of signalling molecules including cAMP, IP3 and free calcium in tissues, tissue slices, intact cells or partially disrupted cells;

ii) use of biological membranes to assess adenosine receptor activation in response to a test compound by the measurement of G protein activation (for example by the use of radiolabelled guanine nucleotides), or by the measurement of enzyme activity (for example adenylyl cyclase, phosphodiesterase, phospholipase or protein kinase), or by the measurement of ion flow through ion channels activated by the adenosine receptor (for example calcium or potassium channels);

iii) determining adenosine receptor action by the measurement of protein kinase activity in tissues, tissue slices, intact cells, disrupted cells or cell derived membranes;

iv) determining adenosine receptor action by the measurement of phospholipase activity (e.g. phospholipase C, phospholipase A2, phospholipase D) in tissues, tissue slices, intact cells, disrupted cells or cell derived membranes;

v) determining adenosine receptor action by the measurement of protein phosphorylation and dephosphorylation in tissues, tissue slices, intact cells, disrupted cells or cell derived membranes.

The level of activity of a compound (i.e. its intrinsic efficacy) can vary depending on the effect desired. For instance, low efficacy agonists may be useful in reducing the probability of receptor desensitisation, or in conferring an extra dimension of targeting, i.e. to those tissues with large receptor reserve. The activity is preferably at least 50%, more preferably at least as great as that for any of the active agents reported below. The difference is preferably seen between physiological pH (7.4) and pH 5.5 or above, e.g. 6.2 (extreme for ischaemic tissue), 6.5, 7.0 (typical in areas of chronic inflammation) or 7.2. Alternatively, low efficacy partial agonists can be used as functional antagonists, binding to the receptor and preventing the binding of the endogenous transmitter/hormone (for example, adenosine).

If a potential therapeutic agent is identified, it may then be determined whether the agent has a therapeutic effect, for example against pain or inflammation (particularly any of the diseases or conditions listed above). Preferably a non human animal model is used to determine whether the identified agent has a therapeutic effect. Any suitable animal model may be used. Examples include arthritis induced by injection of collagen II, or neuropathic pain induced by streptozotocin induced diabetes.

Preferably the therapeutic effect of the identified agent is determined at a concentration below the EC50 value of the agent at the adenosine receptor at pH 7.4. More preferably the therapeutic effect is determined at a concentration that is one ten thousandth to one fifth, or one ten thousandth to one twentieth, or one ten thousandth to one hundredth, most preferably one ten thousandth to one thousandth, of the EC50 value.

To confirm that the identified agent may be administered with minimised side effects, the side effects (such as bradycardia, hypotension or tachycardia) caused by the identified agent may be determined. Preferably these are determined at a concentration below the EC50 value of the agent at the adenosine receptor at pH 7.4. More preferably the side effects are determined at a concentration that is one ten thousandth to one fifth, or one ten thousandth to one twentieth, or one ten thousandth to one hundredth, most preferably one ten thousandth to one thousandth, of the EC50 value.

A number of compounds that are agonists of the adenosine A1 or A2 receptor have been identified. These compounds have higher affinity for the adenosine A1 or A2 receptor at lower pH. It is believed that the receptors change their conformation in response to the changes in pH. The precise mechanism by which this change occurs is unknown, but it is thought to involve histidine residues which have been implicated in agonist binding to these receptors and which have pK values in the physiological range.

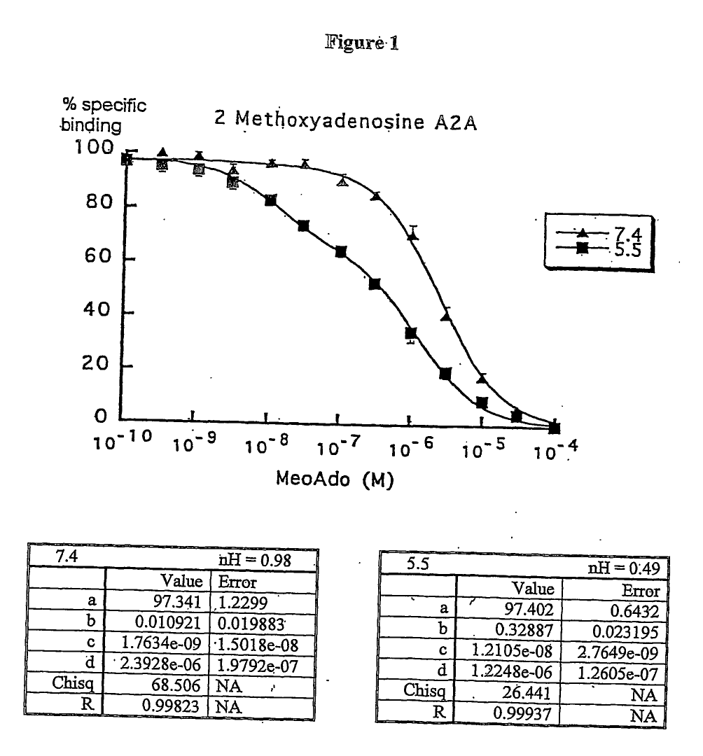

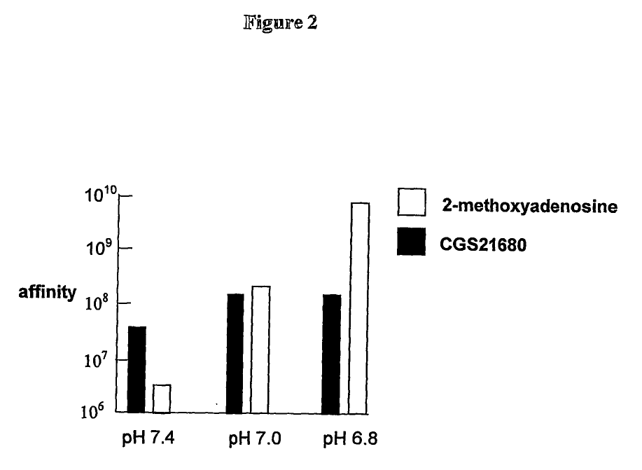

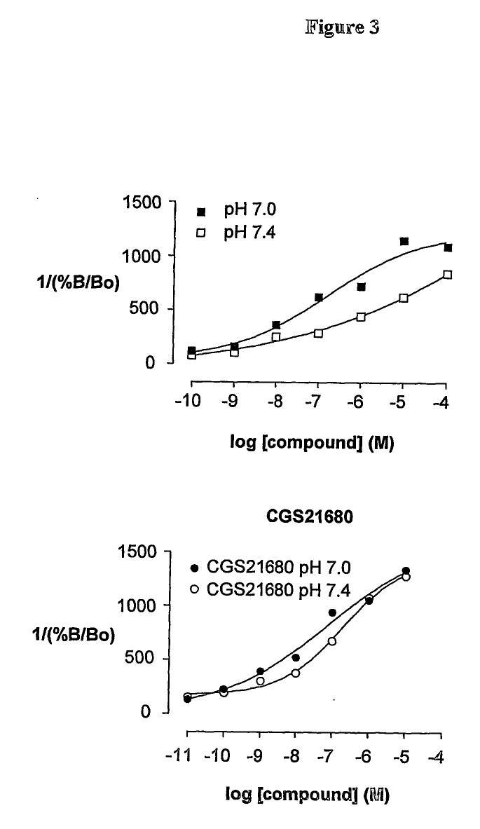

FIG. 1 illustrates the increase in affinity observed with 2-methoxyadenosine as the pH in a ligand binding experiment was reduced from pH 7.4 to pH 5.5. FIG. 2 demonstrates that the same increase in affinity was observed at pH7.0 and pH6.8, but that the archetypal A2A receptor agonist CGS21680 does not show this effect. Another surprising consequence of the affinity changes was that the efficacy of 2-methoxyadenosine at the adenosine A2A receptor was increased approximately 100 fold (FIG. 3). In contrast 3′-deoxy-2-methoxyadenosine showed a decrease in affinity at the A1 receptor when the pH was lowered from 7.4 to 6.2. The Ki values determined for the high affinity sites in the displacement of [3H]-DPCPX from the human A1 receptor were 158±85 nM at pH7.4 and 405±114 nM at pH6.2. Thus, it appears that adenosine receptor agonists and partial agonists can be profoundly affected by local changes in tissue pH and, surprisingly, that the efficacy of these ligands can be either increased or decreased. Compounds that show a decrease in efficacy are likely to act as low efficacy partial agonists.

The applicant has appreciated that potential therapeutic agents may also be identified by administering a test compound in vivo or in vitro at a dose lower than that expected to activate (at pH 7.4) a significant proportion of adenosine receptors (i.e. sufficient adenosine receptors to elicit a beneficial therapeutic effect), and then assessing the difference in the dose required to activate the receptors in the pathological tissue compared to normal tissue.

According to a further aspect of the invention there is provided a method for identifying a potential therapeutic agent which comprises contacting a test compound with an adenosine receptor (an A1, A2A, A2B, or A3 adenosine receptor) in a pathological tissue, cell, or membrane and a corresponding normal tissue, cell, or membrane at a concentration below the EC50 value of the test compound at the adenosine receptor at pH 7.4, and determining whether there is any difference in action of the adenosine receptor in response to contact with the test compound between the normal tissue and the pathological tissue.

Preferably the test compound is contacted with the adenosine receptor at a concentration that is one ten thousandth to one fifth, or one ten thousandth to one twentieth, or one ten thousandth to one hundredth, most preferably one ten thousandth to one thousandth, of the EC50 value.

The test compound is identified as a potential therapeutic agent if the test compound is an agonist of the adenosine receptor, and the action of the adenosine receptor in response to the test compound is greater in the pathological tissue than the normal tissue. If the pathological tissue comprises epithelial tissue, potential therapeutic compounds for the treatment of epithelial disease (for example psoriasis, asthma, COPD) may be identified.

Adenosine receptor agonists that have different receptor affinity and/or efficacy at different pH, or which cause different action of adenosine receptors in pathological tissue compared to normal tissue, include derivatives of adenosine. Thus, it will be appreciated that methods of the invention may be used as screening methods, in particular to screen derivatives of adenosine. Derivatives that have low affinity (i.e. a Kd for an adenosine receptor >0.5 μM) and/or efficacy at pH7.4 are preferred. The selectivity of the derivatives for different adenosine receptors may change at reduced pH, accordingly the selectivity of a given compound should be determined at both normal and reduced pH.

According to the invention compounds of the following general formulae have been found, many of which are believed to have a higher affinity for adenosine receptors, and/or efficacy of action at adenosine receptors, at lower pH: 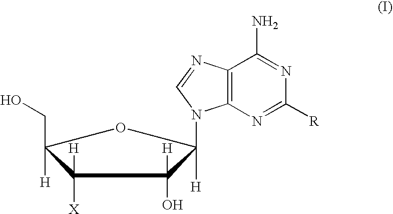
wherein R is C₁₋₄ alkoxy and X is OH; 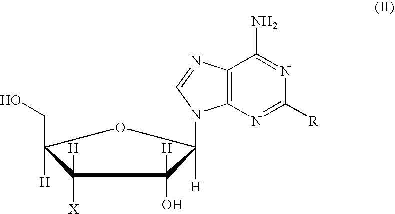
wherein R is C₁₋₄ alkoxy, and X is H.

Compounds exemplified in Example 10 have been found to have a higher affinity for adenosine A2A receptors at lower pH.

Compounds of general formula (I) or (II), or Example 10, that have a higher affinity for adenosine receptors, and/or efficacy of action at adenosine receptors, at lower pH may be used as medicaments in the prevention, treatment, or amelioration of various diseases or conditions. These include diseases or conditions in which prevention, treatment, or amelioration is mediated by activation of adenosine receptors. Examples include diseases or conditions in which local tissue energy demand exceeds supply and/or pH falls.

Compounds of general formula (I) or (II), or Example 10, that have a higher affinity for adenosine receptors, and/or efficacy of action at adenosine receptors, at lower pH may be used in particular for the manufacture of a medicament for the prevention, treatment, or amelioration of cancer, inflammation, ischemia-reperfusion injury, pain, excessive neuronal activity (for example in epilepsy, or chronic pain or hyperalgesia, including inflammatory and neuropathic pain), sepsis, septic shock, neurodegeneration (including Alzheimer's Disease), muscle fatigue or muscle cramp (particularly athletes' cramp).

Compounds of general formula (I) or (II), or Example 10, that have a higher affinity for adenosine receptors, and/or efficacy of action at adenosine receptors, at lower pH may be effective in the prevention, treatment, or amelioration of the following types of pain: bowel pain, pancreatic pain, pelvic/perineal pain, back pain, lower back pain, chest pain, cardiac pain, pelvic pain/PID, joint pain (for example, associated with tendonitis, bursitis, acute arthritis), neck pain, obstetric pain (labour or Caesarean-Section), cancer pain, HIV pain, phantom limb pain; post-operative pain, chronic neuropathic pain, failed back surgery pain, post physical trauma pain (including pain caused by a gunshot wound, a road traffic accident, or a burn), scar tissue pain, acute herpes Zoster pain, acute pancreatitis breakthrough pain (cancer), post-herpes neuralgia, trigeminal neuralgia; neuropathic or other pain caused by, or associated with diabetic neuropathy, polyneuropathy, fibromyalgia, myofascial pain syndrome, osteoarthritis, rheumatoid arthritis, sciatica or lumbar radiculopathy, spinal stenosis, temporo-mandibular joint disorder, renal colic, dysmenorrhoea/endometriosis; or inflammatory or other pain caused by, or associated with arthritic conditions such as osteoarthritis, rheumatoid arthritis, rheumatoid spondylitis, gouty arthritis, or asthma, chronic obstructive pulmonary disease, fibrosis, multiple sclerosis, sepsis, septic shock, endotoxic shock, gram negative shock, toxic shock, hemorrhagic shock, adult respiratory distress syndrome, cerebral malaria, organ transplant rejection, pain secondary to cancer, HIV, chronic pulmonary inflammatory disease, silicosis, pulmonary sarcosis, bone resorption diseases, reperfusion injury, graft v. host rejection, multiple sclerosis, myasthenia gravis, allograft rejections, fever and myalgia due to infection, AIDS related complex (ARC), keloid formation, scar tissue formation, Crohn's disease, ulcerative colitis and pyresis, irritable bowel syndrome, osteoporosis, cerebral malaria, bacterial meningitis, or adverse effects from amphotericin B treatment, interleukin-2 treatment, OKT3 treatment, or GM-CSF treatment.

Compounds of formula (I), compounds of formula (II), and compounds of Example 10, that have a higher affinity for adenosine receptors, and/or efficacy of action at adenosine receptors, at lower pH may be particularly effective for the prevention, treatment, or amelioration of particular types of inflammation, including arthritis (particularly at the joint capsule of arthritis), asthma, psoriasis, and bowel inflammation.

Compounds of formula (I), compounds of formula (II), and compounds of Example 10, that have a higher affinity for adenosine receptors, and/or efficacy of action at adenosine receptors, at lower pH may be particularly effective in the prevention, treatment, or amelioration of rheumatoid arthritis, irritable bowel syndrome or osteoarthritis.

There is further provided according to the invention a method of prevention, treatment, or amelioration of cancer, inflammation, ischemia-reperfusion injury, pain, excessive neuronal activity (for example in epilepsy, or chronic pain or hyperlagesia, including inflammatory and neuropathic pain), sepsis, septic shock, neurodegeneration (including Alzheimer's Disease), muscle fatigue or muscle cramp (particularly athletes' cramp), which comprises administering a compound identified by a method of the invention, or of formula (I) or (II), or of Example 10, that has a higher affinity for adenosine receptors, and/or efficacy of action at adenosine receptors, at lower pH to a subject in need of such prevention, treatment, or amelioration.

Compounds of formula (I) include: 2-methoxyadenosine, 2-ethoxyadenosine, 2-propoxyadenosine, 2-isopropoxyadenosine, and 2-butoxyadenosine. Preferred compounds of formula (I) are 2-methoxyadenosine, 2-ethoxyadenosine, and 2-butyloxyadenosine.

Compounds of formula (II) include: 3′-deoxy-2-methoxyadenosine, 3′-deoxy-2-ethoxyadenosine, 3′-deoxy-2-propoxyadenosine, 3′-deoxy-2-isopropoxyadenosine, and 3′-deoxy-2-butoxyadenosine. Preferred compounds of formula (II) are 3′-deoxy-2-propoxyadenosine, 3′-deoxy-2-isopropoxyadenosine, and 3′-deoxy-2-butoxyadenosine.

2-methoxyadenosine has been reported to have an EC50 value at the adenosine A2A receptor of 3 μM (Daly, J. W. et al. (1993) Pharmacol. 46, 91-100). However, this compound surprisingly has profound anti-hyperalgesic and anti-inflammatory activity at plasma concentrations of 0.2 μM or less. At these low doses 2-methoxyadenosine has reduced probability and severity of side effects. The activity of 2-methoxyadenosine as an analgesic is the subject of International patent application no. PCT/GB03/05379 (unpublished at the filing date of the present application).

Other compounds of formula (I) and compounds of formula (II) (and compounds of Example 10) that have a higher affinity for adenosine receptors, and/or efficacy of action at adenosine receptors, at lower pH (and compounds identified using methods of the invention) are also believed to be much more effective at low doses than other adenosine receptor agonists. Thus, it is expected that such compounds can be effectively administered at doses at which they have reduced probability and severity of side effects. These compounds may alternatively or additionally have reduced probability and severity of side effects compared to other adenosine receptor agonists.

Pain has two components, each involving activation of sensory neurons. The first component is the early or immediate phase when a sensory neuron is stimulated, for instance as the result of heat or pressure on the skin. The second component is the consequence of an increased sensitivity of the sensory mechanisms innervating tissue which has been previously damaged. This second component is referred to as hyperlagesia, and is involved in all forms of chronic pain arising from tissue damage, but not in the early or immediate phase of pain perception.

Thus, hyperalgesia is a condition of heightened pain perception caused by tissue damage. This condition is a natural response of the nervous system apparently designed to encourage protection of the damaged tissue by an injured individual, to give time for tissue repair to occur. There are two known underlying causes of this condition, an increase in sensory neuron activity, and a change in neuronal processing of nociceptive information which occurs in the spinal cord. Hyperalgesia can be debilitating in conditions of chronic inflammation (e.g. rheumatoid arthritis), and when sensory nerve damage has occurred (i.e. neuropathic pain).

Compounds identified by methods of the invention, compounds of formula (I) or (II), and compounds of Example 10, that have a higher affinity for adenosine receptors, and/or efficacy of action at adenosine receptors, at lower pH are believed to be effective in inhibiting pain perception in mammals suffering from neuropathic and inflammatory pain even when administered at doses expected to give concentrations well below those known to activate adenosine receptors. At these doses it is believed that these compounds can treat neuropathic and inflammatory pain without causing the significant side effects associated with administration of other adenosine receptor agonists, and also without reducing normal sensory perception.

Compounds identified by methods of the invention, compounds of formula (I) or (II), of compounds of Example 10, that have a higher affinity for adenosine receptors, and/or efficacy of action at adenosine receptors, at lower pH can be used as anti-hyperalgesics for the prevention, treatment, or amelioration of pain (particularly hyperalgesia) caused as a result of neuropathy, including bowel pain, back pain, cancer pain, HIV pain, phantom limb pain, post-operative pain, diabetic neuropathy, polyneuropathy, post-herpes neuralgia, and trigeminal neuralgia.

Compounds identified by methods of the invention, compounds of formula (I) or (II), or compounds of Example 10, that have a higher affinity for adenosine receptors, and/or efficacy of action at adenosine receptors, at lower pH can be used as anti-hyperalgesics for the prevention, treatment, or amelioration of pain particularly hyperalgesia) caused as a result of inflammatory disease, including bowel pain, back pain, cancer pain, fibromyalgia, post-operative pain, osteoarthritis, and rheumatoid arthritis.

Compounds identified by methods of the invention, compounds of formula (I) or (II), and compounds of Example 10, that have a higher affinity for adenosine receptors, and/or efficacy of action at adenosine receptors, at lower pH are expected to have advantages (such as increased efficacy and/or reduced side effects) when used to treat pain (particularly hyperalgesia) compared to compounds of the two major classes of known analgesics. These are: (i) non steroidal anti-inflammatory drugs (NSAIDs) and the related COX-2 inhibitors; and (ii) opiates based on morphine. Analgesics of both these classes are effective in controlling normal nociceptive pain. However, they are less effective against some types of hyperalgesic pain, such as neuropathic pain. Many medical practitioners are reluctant to prescribe opiates at the high doses required to affect neuropathic pain because of the side effects caused by administration of these compounds, and the possibility that patients may become addicted to them. NSAIDs are much less potent than opiates, so even higher doses of these compounds are required. However, this is undesirable because these compounds cause irritation of the gastrointestinal tract.

The amount of a compound identified by a method of the invention, or of formula (I) or (II), or of Example 10, that is administered to a subject should be an amount which gives rise to a peak plasma concentration that is less than the EC50 value of the compound at adenosine receptors at pH 7.4.

It will be appreciated that the EC50 value of the compound is likely to be different for different adenosine receptors (i.e. the A1, A2A, A2B, A3 adenosine receptors). The amount of the compound that is to be administered should be calculated relative to the lowest EC50 value of the compound at the different receptors.

The peak plasma concentration may be one thousandth to one fifth, or one fiftieth to one third (more preferably one thousandth to one twentieth, one hundredth or one fiftieth to one fifth, one fiftieth to one tenth, or one tenth to one fifth) of the EC50 value. Preferably the peak plasma concentration is one ten thousandth to one fifth, or one ten thousandth to one twentieth, or one ten thousandth to one hundredth, most preferably one ten thousandth to one thousandth of the EC50 value.

Preferably the amount administered gives rise to a plasma concentration that is maintained for more than one hour between one thousandth and one fifth, more preferably between one thousandth and one twentieth, or one hundredth and one fifth, or one fiftieth and one fifth, of the EC50 value of the compound at adenosine receptors at pH 7.4. More preferably the amount administered gives rise to a plasma concentration that is maintained for more than one hour at one ten thousandth to one fifth, or one ten thousandth to one twentieth, or one ten thousandth to one hundredth, most preferably one ten thousandth to one thousandth.

For the avoidance of doubt, the EC50 value of a compound is defined herein as the concentration of the compound that provokes a receptor response halfway between the baseline receptor response and the maximum receptor response (as determined, for example, using a dose-response curve).

The EC50 value should be determined under standard conditions (balanced salt solutions buffered to pH 7.4). For EC50 determinations using isolated membranes, cells and tissues this would be in buffered salt solution at pH 7.4 (e.g. cell culture medium), for example as in Daly et al. (Pharmacol. (1993) 46, 91-100), or preferably Tilburg et al (J. Med. Chem. (2002) 45, 91-100). The EC50 could also be determined in vivo by measuring adenosine receptor mediated responses in a normal healthy animal, or even in a tissue perfused under normal conditions (i.e. oxygenated blood, or oxygenated isotonic media, also buffered at pH 7.4) in a normal healthy animal.

Alternatively, the amount of a compound identified by a method of the invention, or of formula (I) or (II), or of Example 10, that is administered may be an amount that results in a peak plasma concentration that is one thousandth to one twentieth, one thousandth to one third, more preferably one hundredth to one fifth, or one fiftieth to one tenth, of the Kd value at adenosine receptors. More preferably the amount is an amount that results in a peak plasma concentration that is one ten thousandth to one fifth, or one ten thousandth to one twentieth, or one ten thousandth to one hundredth, most preferably one ten thousandth to one thousandth, of the Kd value at adenosine receptors.

It will be appreciated that the Kd value of the compound is likely to be different for different adenosine receptors (i.e. the A1, A2A, A2B, A3 adenosine receptors). The amount of the compound that is to be administered may be calculated relative to the lowest or highest Kd value of the compound for the different receptors.

Preferably the amount of the compound that is administered is an amount that results in a plasma concentration that is maintained for at least one hour between one thousandth and one fifth, more preferably between one thousandth and one twentieth, or one hundredth and one fifth, or one fiftieth and one fifth, of the Kd value of the compound at adenosine receptors. More preferably the amount results in a plasma concentration that is maintained for at least one hour at one ten thousandth to one fifth, or one ten thousandth to one twentieth, or one ten thousandth to one hundredth, most preferably one ten thousandth to one thousandth of the Kd value of the compound at adenosine receptors.

The Kd value of the compound at each receptor should be determined under standard conditions using plasma membranes as a source of the adenosine receptors derived either from tissues or cells endogenously expressing these receptors or from cells transfected with DNA vectors encoding the adenosine receptor genes. Alternatively whole cell preparations using cells expressing adenosine receptors can be used. Labelled ligands (e.g. radiolabelled) selective for the different receptors should be used in buffered (pH7.4) salt solutions (see e.g. Tilburg et al, J. Med. Chem. (2002) 45, 420-429) to determine the binding affinity and thus the Kd of the compound at each receptor.

Alternatively, the amount of a compound identified by a method of the invention, or of formula (I) or (II), or of Example 10, that is administered may be an amount that is one thousandth to one fifth, or one fiftieth to one third (preferably one thousandth to one twentieth, or one hundredth or one fiftieth to one fifth) of the minimum dose of the compound that gives rise to bradycardia, hypotension or tachycardia side effects in animals of the same species as the subject to which the compound is to be administered. Preferably the amount is one tenth to one fifth of the minimum dose that gives rise to the side effects. More preferably the amount is one ten thousandth to one fifth, or one ten thousandth to one twentieth, or one ten thousandth to one hundredth, most preferably one ten thousandth to one thousandth of the minimum does that gives rise to the side effects.

Preferably the amount administered gives rise to a plasma concentration that is maintained for more than 1 hour between one thousandth and one twentieth, or one hundredth or one fiftieth and one fifth of the minimum plasma concentration that gives rise to the side effects. More preferably the amount administered gives rise to a plasma concentration that is maintained for more than 1 hour at one ten thousandth to one fifth, or one ten thousandth to one twentieth, or one ten thousandth to one hundredth, most preferably one ten thousandth to one thousandth, of the minimum plasma concentration that gives rise to the side effects.

Alternatively, the amount of a compound identified by a method of the invention, or of formula (I) or (I), or of Example 10, that is administered may be an amount that gives rise to plasma concentrations that are one thousandth to one fifth, or one fiftieth to one third (preferably one thousandth to one twentieth, or one hundredth or one fiftieth to one fifth) of the minimum plasma concentration of the compound that cause bradycardia, hypotension or tachycardia side effects in animals of the same species as the subject to which the compound is to be administered. Preferably the amount gives rise to plasma concentrations that are one tenth to one fifth of the minimum plasma concentration that causes the side effects. More preferably the amount gives rise to plasma concentrations that are one ten thousandth to one fifth, or one ten thousandth to one twentieth, or one ten thousandth to one hundredth, most preferably one ten thousandth to one thousandth, of the minimum plasma concentration that causes the side effects.

Preferably the amount administered gives rise to a plasma concentration that is maintained for more than 1 hour between one thousandth and one twentieth, or one hundredth or one fiftieth and one fifth, of the minimum plasma concentration that causes the side effects. More preferably the amount gives rise to a plasma concentration that is maintained for more than one hour at one ten thousandth to one fifth, or one ten thousandth to one twentieth, or one ten thousandth to one hundredth, most preferably one ten thousandth to one thousandth, of the minimum plasma concentration that causes the side effects.

The appropriate dosage of a compound identified using a method of the invention, or of formula (I) or (II), or of Example 10, will vary with the age, sex, weight, and condition of the patient, the potency of the compound, and the route of administration, etc. The appropriate dosage can readily be determined by one skilled in the art.

It is expected that the amount of a compound identified by a method of the invention, or of formula (I) or (II), or of Example 10, that is administered should be 0.001 to 15 mg/kg, or 0.01 to 15 mg/kg, for example 0.01 to 5 or 10 mg/kg, or 0.001 to 0.01 mg/kg. The amount may be less than 6 mg/kg, preferably at least 0.001, or 0.01 or 0.05 mg/kg, for example 0.01 to 2 mg/kg. The amount may be at least 0.1 mg/kg, for example 0.1 to 1 or 2 mg/kg, or 0.2 to 1 mg/kg. A typical amount is 0.2 or 0.6 to 1.2 mg/kg.

A unit dosage of a compound identified by a method of the invention, or of formula (I) or (II), or of Example 10, typically comprises up to 500 mg (for example 1 to 500 mg, preferably 5 to 500 mg) of the active agent. Preferably the active agent is in the form of a pharmaceutical composition comprising the active agent and a physiologically acceptable carrier, excipient, or diluent. The preferred dosage is 0.1 to 2, e.g. 0.5 to 1, typically about 0.6, mg of the active agent per kg of the (human) subject. At these levels, effective treatment can be achieved substantially without a concomitant fall (for example, no more than 10%) in blood pressure.

Preferred doses for a 70 kg human subject are less than 420 mg, preferably at least 0.7 mg, more preferably at least 3.5 mg, most preferably at least 7 mg. More preferably 7 to 70 mg, or 14 to 70 mg.

The dosage amounts specified above are significantly lower (up to approximately 1000 times lower) than would be expected (based on the EC50 value of spongosine at the adenosine A2A receptor) to be required for the compounds to have any beneficial therapeutic effect.

Compounds identified by methods of the invention, or compounds of formula (I) or (I), or of Example 10, may be administered with or without other therapeutic agents, for example anti-inflammatories (steroids, NSAIDs, methotrexate), analgesics (such as opiates, NSAIDs, cannabinoids, tachykinin modulators, or bradykinin modulators) or anti-hyperalgesics (such as gabapentin, pregabalin, cannabinoids, sodium or calcium channel modulators, anti-epileptics or anti-depressants).

In general, a compound identified by a method of the invention or a compound of formula (I) or (II), or of Example 10, may be administered by known means, in any suitable formulation, by any suitable route. A compound of the invention is preferably administered orally, parenterally, sublingually, transdermally, intrathecally, or transmucosally. Other suitable routes include intravenous, intramuscular, subcutaneous, inhaled, and topical. The amount of drug administered will typically be higher when administered orally than when administered, say, intravenously.

Suitable compositions, for example for oral administration, include solid unit dose forms, and those containing liquid, e.g. for injection, such as tablets, capsules, vials and ampoules, in which the active agent is formulated, by known means, with a physiologically acceptable excipient, diluent or carrier. Suitable diluents and carriers are known, and include, for example, lactose and talc, together with appropriate binding agents etc.

A preferred administration frequency of compounds of the invention is expected to be two or three times per day.

Compounds of the invention can also serve as a basis for identifying more effective drugs, or drugs that have further reduced side effects.

Embodiments of the invention relating to use of a compound of formula (I) (particularly for the prevention, treatment, or amelioration of inflammation, or pain, such as hyperalgesia) may exclude 2-methoxyadenosine.

Embodiments of the invention relating to compounds of formula (I) may exclude 2-propoxyadenosine, and/or 2-isopropoxyadenosine.

Embodiments of the invention relating to compounds of formula (II) may exclude 3′-deoxy-2-methoxyadenosine and/or 3′-deoxy-2-ethoxyadenosine.

Embodiments of the invention are now described, by way of example only, with reference to the accompanying drawings in which:

FIG. 1 shows the increase in affinity of 2-methoxyadenosine for the adenosine A2A receptor at pH5.5 compared to pH7.4;

FIG. 2 shows that the affinity of 2-methoxyadenosine (but not CGS21680) for the rat adenosine A2A receptor increases as the pH is reduced (from 7.4 to 7.0 to 6.8);

FIG. 3 shows the increase in efficacy of 2-methoxyadenosine, but not CGS21680, for the adenosine A2A receptor at pH7.0 compared to pH7.4;

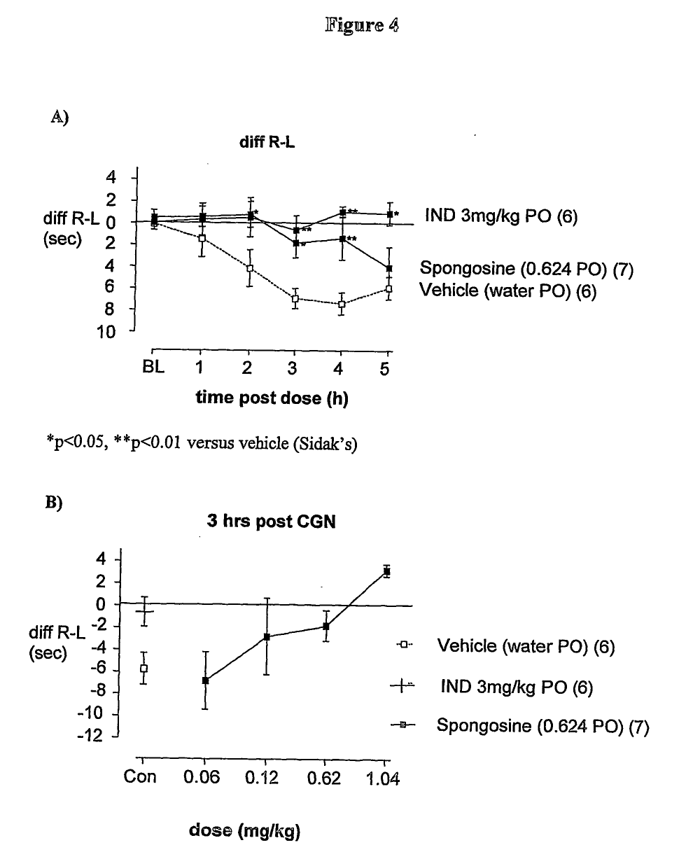

FIG. 4 shows that 2-methoxyadenosine inhibits the hyperalgesic effect of carageenan induced inflammation;

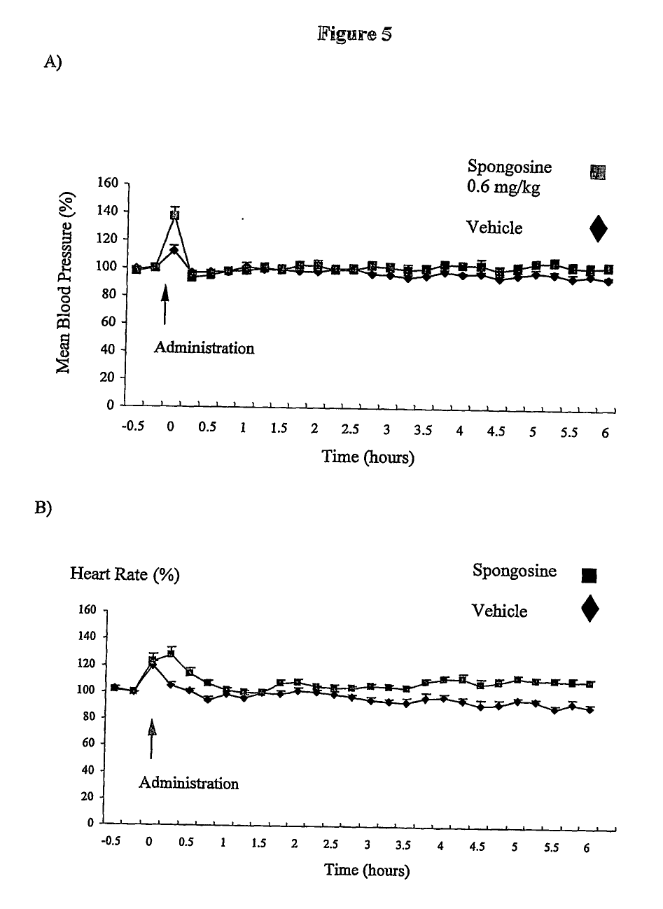

FIG. 5 shows that 2-methoxyadenosine (0.624 mg/kg p.o.) has no significant effect on blood pressure or heart rate;

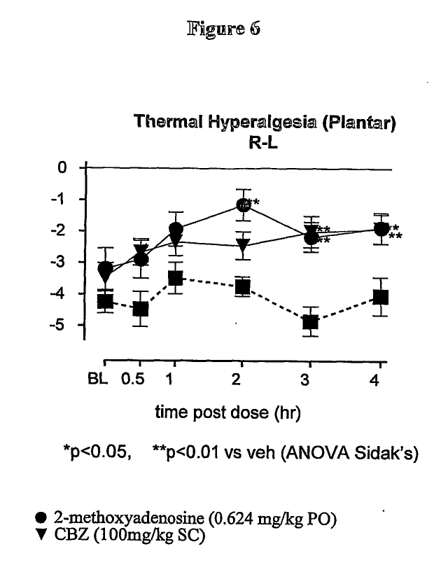

FIG. 6 shows the anti-hyperalgesic action of 2-methoxyadenosine in the chronic constriction injury model of neuropathic pain;

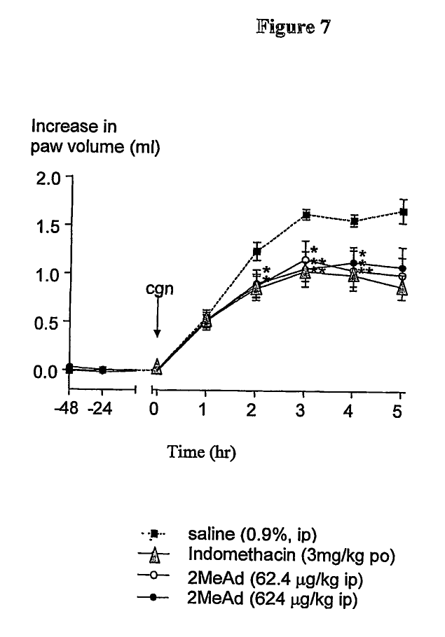

FIG. 7 shows that 2-methoxyadenosine (62.4 and 624 μg/kg i.p.) inhibits carrageenan (CGN) induced inflammation with comparable efficacy to indomethacin (3 mg/kg, po), at concentrations that do not affect blood pressure; and

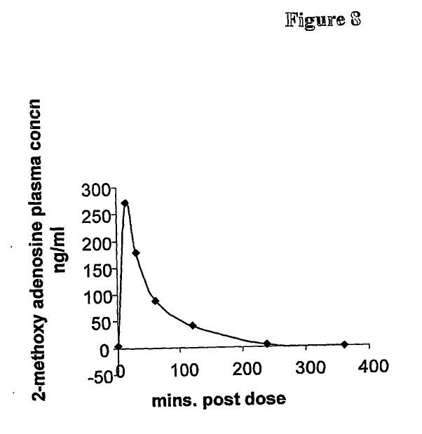

FIG. 8 shows the change in plasma concentration over time after administration of 2-methoxyadenosine (0.6 mg/kg) to a rat.

### EXAMPLE 1

The affinity of 2-methoxyadenosine for the Adenosine A2A Receptor Increases as pH is Reduced

Rat striatal membranes were incubated for 90 minutes at 22° C. in the presence of 2 nM [3H]-CGS21680, 1 Unit/ml adenosine deaminase and increasing concentrations of 2-methoxyadenosine, prior to filtration and liquid scintillation counting. The data were fitted to one and two site binding curves (see FIG. 1):
a) represents the total amount of specific binding as a percentage;
b) represents the proportion of the sites in the high affinity state;
c) represents the Ki of the high affinity state (N.B. at pH 7.4, the Hill slope (nH) was close to unity and the curve fitting algorithm for a two site fit was unable to define realistic properties to a high affinity state); and
d) represents the Ki of the low affinity state.

### EXAMPLE 2

Comparison of the Affinity of 2-methoxyadenosine and CGS21680 for the Adenosine A2A Receptor at Different pH

Displacement of [3H]-CGS21680 binding was performed as described for Example 1. The results, shown in FIG. 2, demonstrate that the affinity of 2-methoxyadenosine (but not CGS21680) for the rat adenosine A2A receptor increases as the pH is reduced (from 7.4 to 7.0 to 6.8).

### EXAMPLE 3

The efficacy of 2-methoxyadenosine, but not CGS21680, for the adenosine A2A Receptor is Increased at pH7.0 Compared to pH7.4

The human A2A receptor was expressed in HEK293 cells, and the ability of agonists to stimulate cAMP accumulation assessed in the presence of rolipram to inhibit phosphodiesterase enzymes. The results are shown in FIG. 3.

### EXAMPLE 4

2-methoxyadenosine Inhibits the Hyperalgesic Effect of Carageenan Induced Inflammation

FIG. 4 shows that 2-methoxyadenosine inhibits the hyperalgesic effect of carageenan induced inflammation: A. 2-methoxyadenosine (0.6 mg/kg) inhibits carrageenan (CGN) induced thermal hyperalgesia (CITH) with comparable efficacy to indomethacin (3 mg/kg, po). B. Concentration-response relationship for 2-methoxyadenosine at 3 hrs post dosing.

Carrageenan (2%, 10 microlitres) was administered into the right hind paw. A heat source was placed close to the treated and untreated hind paws, and the difference in the paw withdrawal latencies is shown. 2-methoxyadenosine was administered at the same time as carrageenan. 2-methoxyadenosine was as effective as indomethacin (Indo, 3 mg/kg p.o.).

### EXAMPLE 5

2-methoxyadenosine (at 0.624 mg/kg p.o.) has no Significant Effect on Blood Pressure or Heart Rate

An implantable radiotelemetry device was placed in the abdominal cavity of 6 rats per group. The pressure catheter of the device was inserted in the abdominal aorta and two electrodes tunnelised under the skin in a lead II position (left side of abdominal cavity/right shoulder). Individual rats were placed in their own cage on a radioreceptor (DSI) for data acquisition. The effect of 0.6 mg/kg 2 methoxyadenosine or vehicle (p.o.) on blood pressure was then assessed. The results are shown in FIG. 5: A: blood pressure; B: heart rate.

### EXAMPLE 6

The Anti-Hyperalgesic Action of 2-methoxyadenosine in the Chronic Constriction Injury Model of Neuropathic Pain

2-methoxyadenosine (0.624 mg/kg p.o.) inhibits thermal hyperalgesia caused by chronic constriction injury of the rat sciatic nerve. Under anaesthesia the sciatic nerve was displayed in the right leg, and four loose ligatures tied round the nerve bundle. After approximately two weeks the rats developed thermal hyperalgesia in the operated leg as judged by the difference in paw withdrawal latencies of the right and left paws. Administration of 2-methoxyadenosine reduced the hyperalgesia as shown by the reduction in the difference between the withdrawal latencies. 2-methoxyadenosine was as, or more, effective than carbamazepine (CBZ, 100 mg/kg s.c.). The results are shown in FIG. 6.

### EXAMPLE 7

2-methoxyadenosine (62.4 and 624 μg/kg i.p.) Inhibits Carrageenan (CGN) Induced Inflammation with Comparable Efficacy to Indomethacin (3 mg/kg, po), at Concentrations that do not Affect Blood Pressure

Carrageenan (2%, 10 microlitres) was administered into the right hind paw, and the paw volume assessed by plethysomometry. 2-methoxyadenosine was administered at the same time as carrageenan. The results are shown in FIG. 7. 2-methoxyadenosine was as effective as indomethacin (Indo, 3 mg/kg p.o.).

### EXAMPLE 8

The Change in Plasma Concentration Over Time after Administration of 2-methoxyadenosine (0.6 mg/kg) to a Rat

The EC50 value of 2-methoxyadenosine at adenosine receptors (measured at pH7.4) is 900 ng/ml (3 μM). It can be seen from FIG. 8 that the plasma concentration remains above 2% of the EC50 value for more than 3 hours. Anti-inflammatory and anti-hyperalgesic effects have been observed (without blood pressure changes) when the peak plasma concentration is between 1% and 30% of the EC50 value determined in vitro. If the peak plasma concentration reaches the EC50 value profound reductions in blood pressure occur that last for hours.

### EXAMPLE 9

The increased affinity of adenosine receptor agonists at lower pH was associated with a corresponding increase in GTP shift, suggesting that these agonists were also more efficacious at the lower pH. These results are summarised below.

TABLE 1
Displacement of 3H-CGS21680 from the rat striatal A2A receptor. (values
are Ki in nM)

| | pH 7.4 | pH5.5 |
|---|---|---|
| CGS21680 | 10.5 ± 2.1 | 2.8 ± 1.0 |
| NECA | 3.5 ± 0.9 | 1.1 ± 0.2 |
| CV1808 | 170 ± 20 | 35 ± 9 |
| R-PIA | 172 ± 21 | 21 ± 1.4 |
| S-PIA | 1712 ± 423 | 271 ± 4.9 |

NECA: 5′ N-ethylcarboxamidoadensosine;
CV1808: 2-phenylamino-adenosine;
PIA: phenylisopropyladenosine.

All of the above revealed only one affinity state for agonists, suggesting that the high and low affinity states are similar in affinity. However 2-methoxyadenosine and 2-ethoxyadenosine revealed two apparent affinity states at the lower pH (Table 2). The high affinity states corresponded to approximately 25% of the total number of binding sites, and were abolished by the presence of GTP, suggesting that they correspond to the high affinity agonist binding state.

TABLE 2
Displacement of 3H-CGS21680 from the rat striatal A2A receptor. (values
are Ki in nM)
2-methoxyadenosine

| | | |
|---|---|---|
| pH 7.4: | High affinity not detectable | Low affinity 2500 ± 200 |
| pH 5.5: | High affinity 12.1 ± 2.7 | Low affinity 1200 ± 100 |

2-ethoxyadenosine

| | | |
|---|---|---|
| pH 7.4: | High affinity 245 ± 76 | Low affinity 6600 ± 2200 |
| pH 5.5: | High affinity 2.8 ± 1.2, | Low affinity 1200 ± 120 |

In addition compounds were discovered which showed higher affinity for the rat A1 receptor at lower pH (Table 3).

TABLE 3
Displacement of 3H-DPCPX from the rat cortex A1 receptor (Ki nM).
2-chloroadenosine

| | | |
|---|---|---|
| pH 7.4 | High affinity 4.4 ± 1.8 | Low affinity 1670 ± 346 |
| pH 6.2 | High affinity 3.7 ± 0.5 | Low affinity 1660 ± 40 |

3′-deoxy-2-chloroadenosine

| | | |
|---|---|---|
| pH 7.4: | High affinity 227 ± 74 | Low affinity 11,500 ± 850 |
| pH 6.2: | High affinity 17 ± 46 | Low affinity 13,400 ± 2400 |

At both pH values the stable analogue of GTP (GppNHp) abolished the high affinity state recognised by 3′-deoxy-2-chloroadenosine.

### EXAMPLE 10

Compounds Found to Have Higher Affinity for Adenosine A2A Receptors at Lower pH

The compounds listed below were found to have higher affinity for adenosine A2A receptors at pH 5.5 than pH7.4. The A2A receptor binding assay was carried out using [3H]-CGS21680 and analysed as described for Example 1. The Ki values of the high and low affinity binding sites detected at pH5.5 are indicated, and the Ki of the site detected at pH7.4.

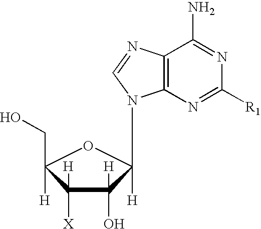

| | | | |
|---|---|---|---|
| Structure | pH 5.5 | pH 5.5 | pH 7.4 |
| R₁ | (Ki 1) nM | (Ki 2) nM | (Ki 2) nM |

When X = OH

| | | | |
|---|---|---|---|
| OCH3 | 1.5 | 380 | 1300 |
| OCH₂CH₂CH₂CH₃ | 11 | 560 | 280 |
| O CH₂CH₂CH₂CH₂CH₂CH₃ | 3 | 170 | 1500 |
| OPh | 71 | 1200 | 2500 |
| O-(4-cyano)Ph | 4 | 380 | 1300 |
| O-(3-Ph)Ph | 0.7 | 135 | 620 |
| 5-indanyloxy | 12 | 175 | 760 |
| O-(3-CH(CH₃)₂)Ph | 16 | 240 | 560 |
| NH(CH₃) | 24 | 2240 | 1356 |
| NHCH₂CH₃ | 130 | 3500 | 1200 |
| N(CH₃)₂ | 24 | 21440 | 13350 |
| NHCH₂CH₂CH₂CH₂CH₂CH₃ | 0.7 | 20 | 290 |
| NHPh | 5 | 2028 | 160 |
| NH-(4-MeO)Ph | 3 | 180 | 55 |
| NH-(4-F)Ph | 10 | 150 | 200 |
| NH-cyclopentyl | 2 | 60 | 420 |
| NH-cyclohexyl | 0.4 | 100 | 1000 |
| N—CH₃, N—CH₂CH₂CH(CH₃)₂ | 26 | 2600 | 4000 |
| OCH₂cyclopentyl | 0.2 | 54 | 200 |
| SO₂CH₂CH₃ | 100 | 5250 | 39000 |
| OCH₂CH₂OH | 4 | 164 | 203 |
| CH₂CH₂CH₂CH₂CH₂CH₂CH₃ | 15 | 630 | 800 |

X = H

| | | | |
|---|---|---|---|
| O CH₂CH₂CH₂CH₂CH₂CH₃ | 13 | 440 | 2990 |

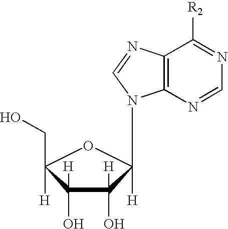

| Structure | pH 5.5 | pH 5.5 | pH 7.4 |
|---|---|---|---|
| R₂ | (Ki 1) nM | (Ki 2) nM | (Ki 2) nM |
| N(CH₃)₂ | 42 | 9400 | 450000 |
| NHCH₂CHC(CH₃)₂ | 1.5 | 380 | 8600 |
| N—CH₃, N—CH₂Ph | 7 | 2100 | 18500 |
| Piperazinyl | 38 | 7000 | 5000 |
| N—Me, N—(CH₂CH₂OCH₃) | 13 | 6470 | 13000 |

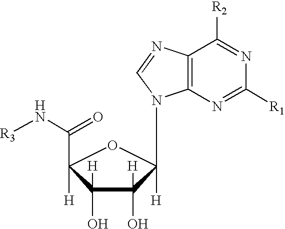

| | | | pH 5.5 | pH 5.5 | pH 7.4 |
|---|---|---|---|---|---|
| R₁ | R₂ | R₃ | (Ki 1) nM | (Ki 2) nM | (Ki 2) nM |
| H | NH₂ | CH(CH₃)₂ | 5 | 190 | 1930 |
| H | NH₂ | H | 9 | 180 | 270 |
| H | NHCH₃ | CH(CH₃)₂ | 188 | 3420 | 2440 |
| OCH₃ | NH₂ | Ph | 230 | 53400 | 26100 |

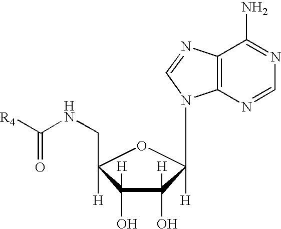

| Structure | pH 5.5 | pH 5.5 | pH 7.4 |
|---|---|---|---|
| R₄ | (Ki 1) nM | (Ki 2) nM | (Ki 2) nM |
| CH₂CH₂CH₃ | 145 | 53550 | 16900 |
| NHCH₂CH₃ | 40 | 5900 | 6570 |

1. A method for identifying a potential therapeutic agent, which comprises determining the affinity of a test compound for an adenosine receptor at a higher pH of at least 7.4, and also at a lower pH from 5.5 to 7.2, and identifying the compound as a said agent if the affinity at the lower pH is over 10 times greater than the affinity at the higher pH.

2. A method according to claim 1 wherein determining the affinity of the test compound comprises measuring binding of different concentrations of labelled test compound to the receptor.

3. A method according to claim 1 wherein determining the affinity of the test compound comprises measuring displacement of a bound labelled compound from the adenosine receptor with unlabelled test compound.

4. A method according to claim 1, wherein the adenosine receptor is in a tissue, a tissue slice, an intact cell, a disrupted cell, or a cell derived membrane.

5. A method for identifying a potential therapeutic agent, which comprises determining the efficacy of a test compound at an adenosine receptor at a higher pH of at least 7.4, and also at a lower pH from 5.5 to 7.2, and identifying the compound as a said agent if the efficacy at the lower pH is over 10 times greater than the efficacy at the higher pH.

6. A method according to claim 5, wherein determining the efficacy of the test compound comprises contacting the test compound with a tissue, tissue slice, intact cell or partially disrupted cell comprising the adenosine receptor, and determining adenosine receptor action by measuring accumulation or depletion of a signalling molecule in the tissue, tissue slice, intact cell or partially disrupted cell.

7. A method according to claim 6 wherein the signalling molecule is cAMP, IP3 or free calcium.

8. A method according to claim 5, wherein determining the efficacy of the test compound comprises contacting the test compound with a biological membrane comprising the adenosine receptor, and determining adenosine receptor action by measuring: activation of a G protein; activity of an enzyme; or ion flow through an ion channel associated with the biological membrane.

9. A method according to claim 8, wherein activation of the G protein is measured by use of a radiolabelled guanine nucleotide, or wherein the enzyme activity measured is adenylyl cyclase, phosphodiesterase, phospholipase or protein kinase activity, or wherein the ion channel through which ion flow is measured is a calcium or a potassium channel.

10. A method according to claim 5, wherein determining the efficacy of the test compound comprises contacting the test compound with a tissue, tissue slice, intact cell, disrupted cell or cell derived membrane comprising the adenosine receptor, and determining adenosine receptor action by measuring protein kinase activity in the tissue, tissue slice, intact cell, disrupted cell or cell derived membrane.

11. A method according to claim 5, wherein determining the efficacy of the test compound comprises contacting the test compound with a tissue, tissue slice, intact cell, disrupted cell or cell derived membrane comprising the adenosine receptor, and determining adenosine receptor action by measuring phospholipase activity (e.g. phospholipase C, phospholipase A2, phospholipase D) in the tissue, tissue slice, intact cell, disrupted cell or cell derived membrane.

12. A method according to claim 5, wherein determining the efficacy of the test compound comprises contacting the test compound with a tissue, tissue slice, intact cell, disrupted cell or cell derived membrane comprising the adenosine receptor, and determining adenosine receptor action by measuring protein phosphorylation and/or dephosphorylation in the tissue, tissue slice, intact cell, disrupted cell or cell derived membrane.

13. A method according to claim 1, wherein the lower pH is from 5.5 to 6.5.

14. A method according to claim 1, wherein the lower pH is from 5.5 to 6.2.

15. A method according to claim 1, wherein the lower pH is from 5.0 to 7.0 rather than 5.5 to 7.2.

16. A method according to claim 1, wherein the adenosine receptor is an adenosine A2A receptor, an adenosine A1 receptor, or an adenosine A3 receptor.

17. A method according to claim 1, which further comprises determining whether the identified agent has a therapeutic effect.

18. A method according to claim 17 in which a non human animal model is used to determine whether the identified agent has a therapeutic effect.

19. A method according to claim 17 or 18, wherein it is determined whether the identified agent has a therapeutic effect against pain or inflammation.

20. A method according to claim 17, wherein it is determined whether the identified agent has a therapeutic effect against cancer, ischemia-reperfusion injury, excessive neuronal activity (for example in epilepsy, or chronic pain or hyperlagesia, including inflammatory and neuropathic pain), sepsis, septic shock, neurodegeneration (including Alzheimer's Disease), muscle fatigue or muscle cramp.

21. A method according to claim 17, wherein it is determined whether the identified agent has a therapeutic effect against arthritis, asthma, psoriasis, bowel inflammation, rheumatoid arthritis, irritable bowel syndrome or osteoarthritis.

22. A method according to claim 17, wherein it is determined whether the identified agent has a therapeutic effect against hyperalgesia caused as a result of neuropathy, including bowel pain, back pain, cancer pain, HIV pain, phantom limb pain, post-operative pain, diabetic neuropathy, polyneuropathy, post-herpes neuralgia, or trigeminal neuralgia.

23. A method according to claim 17, wherein it is determined whether the identified agent has a therapeutic effect against hyperalgesia caused as a result of inflammatory disease, including bowel pain, back pain, cancer pain, fibromyalgia, post-operative pain, osteoarthritis, or rheumatoid arthritis.

24. A method according to claim 17 in which the therapeutic effect of the identified agent is determined at a concentration below the EC50 value, preferably one ten thousandth to one fifth of the EC50 value, of the agent at the adenosine receptor at pH 7.4.

25. A method according to claim 17 in which the side effects caused by the identified agent are determined.

26. A method according to claim 25 in which the side effects are determined at a concentration below the EC50 value, preferably one ten thousandth to one fifth of the EC50 value, of the agent at the adenosine receptor at pH 7.4.

27. A method according to claim 25 in which the side effects are bradycardia, hypotension or tachycardia side effects.

28. A method for identifying a potential therapeutic agent which comprises contacting a test compound with an adenosine receptor in a pathological tissue, cell, or membrane and a corresponding normal tissue, cell, or membrane at a concentration below the EC50 value of the test compound at the adenosine receptor at pH 7.4, and determining whether there is any difference in action of the adenosine receptor in response to contact with the test compound between the normal tissue and the pathological tissue.

29. A method according to claim 28, wherein the test compound is identified as a potential therapeutic agent if the test compound is an agonist of the adenosine receptor, and the action of the adenosine receptor in response to the test compound is greater in the pathological tissue than the normal tissue.

30. A method according to claim 28, wherein the pathological tissue comprises epithelial tissue.

31. A method according to claim 30 for identifying a potential therapeutic agent for treating psoriasis, asthma, or COPD.

32. A potential therapeutic agent identified by a method according to claim 28.

33. A potential therapeutic agent according to claim 32 for use as a medicament.
